(19) United States
(12) Patent Application Publication
(10) Pub. No.: US 2014/0207242 A1
(43) Pub. Date: Jul. 24, 2014

(54) System and Method for Hemi Knee Replacement for Tibial Plateau Fracture

(71) Applicant: Mark Vrahas, Newton, MA (US)

(72) Inventor: Mark Vrahas, Newton, MA (US)

(21) Appl. No.: 14/160,611

(22) Filed: Jan. 22, 2014

Related U.S. Application Data
(60) Provisional application No. 61/755,146, filed on Jan. 22, 2013.

(51) Int. Cl. A61F 2/38

(52) U.S. Cl. CPC A61F 2/389
USPC 623/20.15

(57) ABSTRACT

The present invention provides a prosthetic implant for partial replacement of an anatomical part. The implant includes a unitary body with a generally flat lower end face for supporting the body on a cortical surface, a generally curved exterior side face and a generally flat upper end face. A modular bearing plate disposed on the upper end face includes an upper articular surface for supporting articulation of an anatomical surface and an opposed mounting surface in contact with the upper end face. A fixation bracket includes a first end coupled to the exterior side face of the body and a second end projecting downward past the lower end face of the body with a contour generally corresponding to an outer surface of an anatomical part. The prosthetic implant is configured to replace a portion of the outer surface of the anatomical part and extend partially into the anatomical part.

### CROSS-REFERENCE TO RELATED APPLICATIONS

This application is based on, claims the benefit of, and incorporates herein by reference U.S. Provisional Application No. 61/755,146, filed Jan. 22, 2013.

### STATEMENT REGARDING FEDERALLY SPONSORED RESEARCH

Not applicable.

### BACKGROUND OF THE INVENTION

The disclosure relates, in general, to prosthetic devices for partial replacement of an anatomical part and, more particularly, to a system and method for a prosthetic implant for treating a tibial plateau fracture.

Pathologic bone fractures occur as the result of diseases affecting bone strength. Typically, the bone is weakened due to osteoporosis; however, other diseases such as cancer, infection, inherited bone disorders and bone cysts can lead to pathologic bone fractures as well. When a pathologic fracture occurs as the result of normal activities, such as a fall from standing height or less, the fracture can be classified as a fragility fracture. Although fragility fractures are usually associated with the hip and wrist, an ever increasing number of tibial plateau fractures are being reported in elderly patients. Currently, tibial plateau fractures make up about 8% of fractures occurring in the elderly, and almost all of these fractures require surgery.

With respect to treatment of tibial plateau fractures, one aspect of achieving a positive result and preventing rapid deterioration of the knee is to regain alignment of the joint by elevating the depressed portion of the tibial plateau. An emphasis can be placed on providing a sound structural platform to prevent the knee from collapsing into varus or valgus deformity, with secondary consideration given to maintaining the congruency of the joint.

Difficulties associated with treating tibial plateau fractures can be at least partially attributed to nature of the osteoporotic bone. Because the bone is soft, even a low energy fracture can destroy the surface of the tibia leaving no sizable pieces remaining to rebuild the joint. Even if it is possible to restore the pieces to their original locations, the soft bone makes if difficult to achieve proper fixation. In one aspect, the fixation can be so weak that the patient cannot bear weight on the leg for several months as the application of excessive force to joint, even after restoration with fixation plates and screws can result in subsequent collapse/failure of the previously damaged bone. Furthermore, if a patient lacks sufficient upper body strength to use a walker or other mobility aid, then the patient may become confined to their bed for a prolonged period of time.

While there are several procedures available to a surgeon for treating fragility fractures, each possesses a number of drawbacks. For example, conventional knee arthroplasties are not designed for situations where there has been isolated damage to the knee joint as occurs with a fracture. Revision knee arthroplasty components are designed to replace damaged bone, but these require extensive resection of the femur and the tibia. In fracture patients this would require removal of a massive amount of otherwise normal bone and joint, so acute knee replacement is often avoided for fracture patients. Therefore, there remains a need for an improved prosthetic device and method of treatment for pathologic bone fractures. In particular, there is a need for a system and method to treat tibial plateau fractures that overcomes the aforementioned drawbacks of current treatment methods and devices.

### SUMMARY OF THE INVENTION

The present disclosure overcomes the aforementioned drawbacks by providing a prosthetic device designed to at least partially replace the damaged bone and joint resulting from a fragility fracture rather than to repair it. The device creates immediate structural integrity and enables immediate weight-bearing capabilities for the patient. Furthermore, the device is designed to integrate with the native bone and promote osteogenesis to provide a long term solution.

In one embodiment, the prosthetic device is designed with a porous metal body that approximates trabecular bone to replace the damaged bone and allow in-growth of the patient's intact bone. For treatment of tibial plateau fractures, the surface of this body is coupled to a modular cobalt-chrome surface that can replace the articular cartilage. The meniscus and ligaments can be preserved, so only a small amount of the cobalt-chrome would be exposed to the femoral cartilage. In the case where a patient develops arthritis or another condition requiring further treatment of the knee joint at a later date, a standard knee replacement can be implanted without removing the device. Instead, the modular surface of the device can be removed to enable implantation of a total knee replacement device.

In accordance with one aspect of the present disclosure, a prosthetic implant for partial replacement of an anatomical part is provided. The implant includes a unitary body with a generally flat lower end face for supporting the body on a cortical surface, a generally curved exterior side face and a generally flat upper end face. A modular bearing plate disposed on the upper end face includes an upper articular surface for supporting articulation of an anatomical surface and an opposed mounting surface in contact with the upper end face. A fixation bracket includes a first end coupled to the exterior side face of the body and a second end projecting downward past the lower end face of the body with a contour generally corresponding to an outer surface of an anatomical part. The prosthetic implant is configured to replace a portion (e.g., less than all) of the outer surface of the anatomical part and extend partially into the anatomical part.

In accordance with another aspect of the present disclosure, a knee joint prosthetic implant for partial replacement of a proximal portion of the tibia is provided. The knee joint includes a unitary body with a generally flat lower end face for supporting the body on a cortical surface of the tibia, a generally curved exterior side face and a generally flat upper end face. A modular bearing plate is disposed on the upper end face, and has an upper articular surface for supporting articulation of a condyle surface of a femur and an opposed mounting surface in contact with the upper end face. A fixation bracket has a first end coupled to the exterior side face and a second end projecting downward past the lower end face of the body with a contour generally corresponding to an outer surface of the tibia. The prosthetic implant is configured to replace a portion of the proximal end of the tibia including at least a portion (e.g., less than all) of the tibial plateau, and extend partially into the tibia.

In accordance with yet another aspect of the disclosure, a method of implanting a prosthetic implant for partial replacement of an anatomical part is provided. The method includes the steps of resecting a portion (e.g., less than all) of the anatomical part, implanting a unitary body in the resected portion, the body including a generally flat lower end face for supporting the body on a cortical surface of the anatomical part, a generally curved exterior side face and a generally flat upper end face, supporting a modular bearing plate on the body, the modular bearing plate disposed on the upper end face, the bearing plate having an upper articular surface for supporting articulation of an anatomical surface and an opposed mounting surface in contact with the upper end face, and fixing the body to the anatomical part with a fixation bracket, the fixation bracket having a first end and an opposed second end, the first end of the fixation bracket coupled to the exterior side face and the second end projecting downward past the lower end face of the body, the fixation bracket having a contour generally corresponding to an outer surface of the anatomical part. The prosthetic implant is configured to replace a portion of the outer surface of the anatomical part and extend partially into the anatomical part.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is an anterior view of the left knee joint including a non-limiting example of a prosthetic device according to the present disclosure.

FIG. 2 is a lateral view of the of the left knee joint including the prosthetic device of FIG. 1.

FIG. 3 is a front view showing the interior face of the prosthetic device of FIG. 1 in isolation.

FIG. 4 is a side view of the prosthetic device of FIG. 3.

FIG. 5 is a rear view of the prosthetic device of FIG. 3.

FIG. 6 is a top view of the prosthetic device of FIG. 3.

FIG. 7 is a front view of a body of the prosthetic device of FIG. 3 in isolation.

FIG. 8 is a side view of the body of FIG. 7.

FIG. 9 is a top view of the body of FIG. 7.

FIG. 10 is a rear view of the body of FIG. 7.

FIG. 11 is a cross-section view taken along the line 11-11 of FIG. 10.

FIG. 12 is a front view of a mounting bracket of the prosthetic device of FIG. 3 in isolation.

FIG. 13 is side view of the mounting bracket of FIG. 12.

FIG. 14 is rear view of the mounting bracket of FIG. 12.

FIG. 15 is cross-section view taken along the line 15-15 of FIG. 14.

FIG. 16 is a side view of a modular bearing plate of the prosthetic device of FIG. 3.

FIG. 17 is a rear view of the modular bearing plate of FIG. 16.

FIG. 18 is a top view of the modular bearing plate of FIG. 16.

FIG. 19 is a bottom view of the modular bearing plate of FIG. 16.

FIG. 20 is perspective view of the prosthetic device of FIG. 3.

Like reference numerals will be used to refer to like parts from figure to figure in the following detailed description.

### DETAILED DESCRIPTION OF THE INVENTION

The present invention is presented in several varying embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the system. One skilled in the relevant art will recognize, however, that the system and method may both be practiced without one or more of the specific details, or with other methods, components, materials, and so forth. In other instances, well-known structures, materials, or operations are not shown or described in detail to avoid obscuring aspects of the invention.

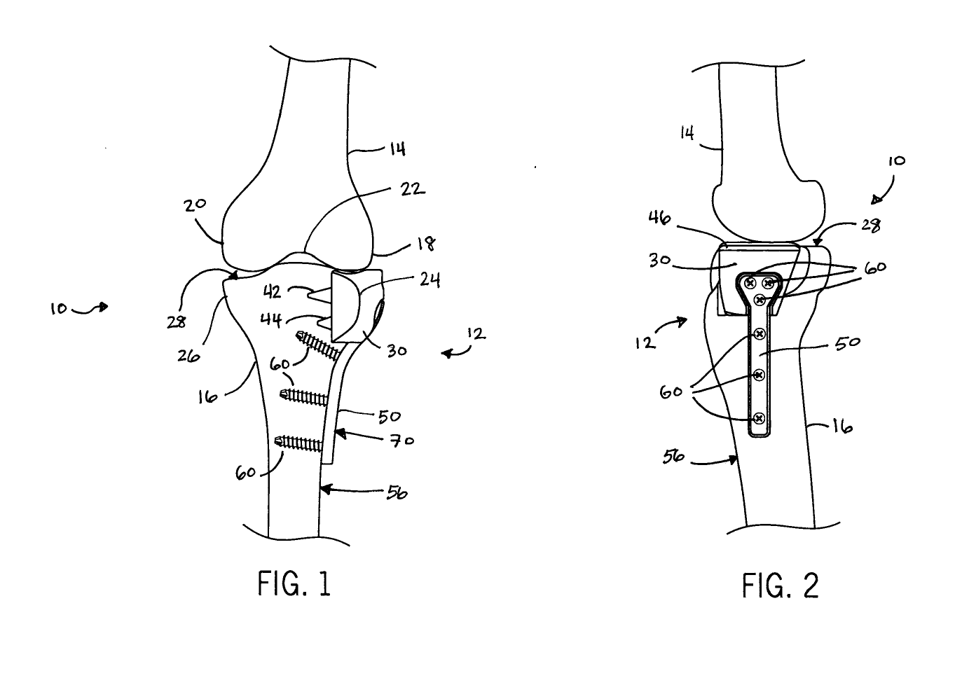

The present disclosure is best understood with reference to the Figures. A prosthetic device according to the present disclosure can be used in the treatment of any suitable anatomical part. In one aspect, embodiments of the present disclosure are generally useful for treating pathologic bone fractures and, more particularly, for treating fragility bone fractures. Examples of fragility bone fractures include vertebral, neck, femur, and wrist fractures. In one preferred embodiment illustrated in FIGS. 1-2, the prosthetic device is used in the treatment of a tibial plateau fracture.

With reference to FIG. 1-2, a left knee joint 10 including an example prosthetic device 12 is schematically illustrated. The knee joint 10 includes a distal end of a femur 14 and a proximal end of a tibia 16. While the schematic illustration of knee joint 10 has been simplified with several components removed for clarity, features of the femur 14 can be seen to include the lateral condyle 18, medial condyle 20 and intercondyloid fossa 22. The tibia 16 includes a partial lateral condyle 24 and medial condyle 26. The upper (proximal) extremity of the tibia 16 defines that tibial plateau 28, which includes portions of the lateral and medial condyles 24, 26. In the example embodiment illustrated in FIGS. 1-2, a portion of the proximal end of the tibia 16 including a portion of the lateral condyle 24 has been resected in order to accommodate the prosthetic device 12.

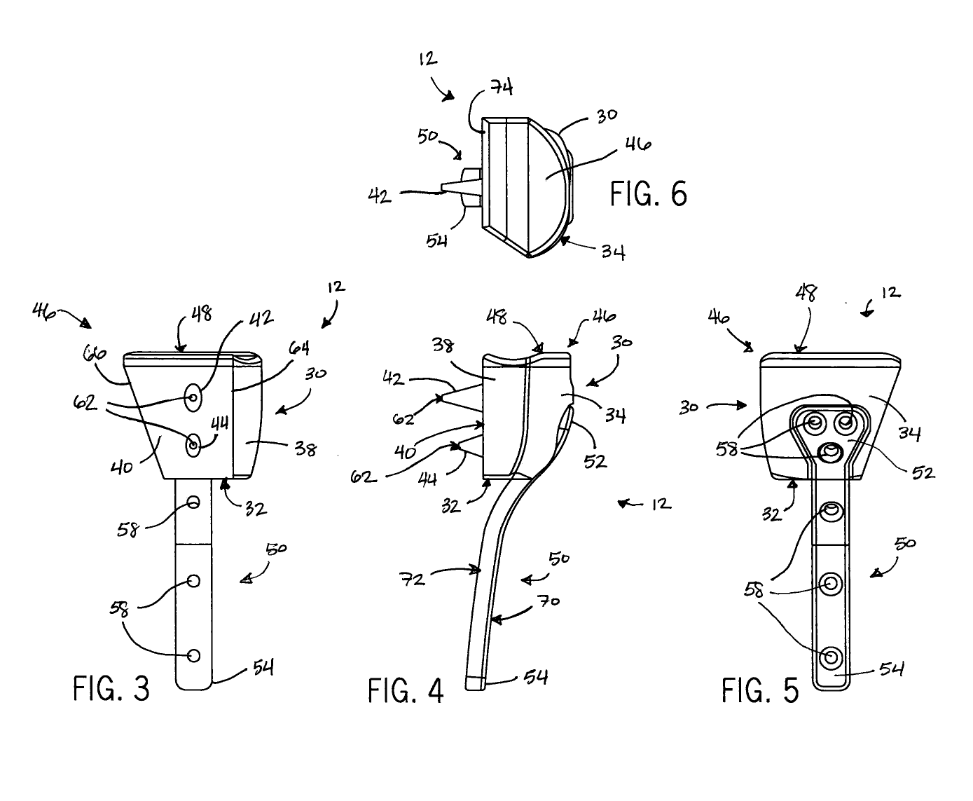

Referring now to FIGS. 3-6 and 20, the prosthetic device 12 according to the present example includes a body 30 with a generally flat lower end face 32, a curved exterior side face 34, a generally flat upper end face 36 and generally flat interior faces 38 and 40. A pair of conical projections 42, 44 extends orthogonally outward from interior face 49. The conical projections 42, 44 are arranged generally parallel to each other as well as to the surface of the upper end face 36, and taper away from the interior surface 40 such that a cross-sectional diameter of the projections 42, 44 is greater at a point proximal to the interior surface 40 than at a point distal to the interior surface 40.

A modular bearing plate 46 is disposed on the upper end face 36 of the body 30. The modular bearing plate 46 includes an upper articular surface 48 for supporting articulation of an articulating surface of the distal end of the femur 14, and in particular, a surface of the lateral condyle 18. A mounting surface 49 is opposed to the articular surface 48 and is in contact with the upper end face 36. In the present embodiment, the modular bearing plate 46 and more particularly the mounting surface 49 has a surface dimension that matches the upper end face 36 such that the mounting surface 49 and body 30 share a perimeter at the interface between these two components of the prosthetic device 12.

With continued reference to FIGS. 3-6, a fixation plate or bracket 50 includes a first end 52 and an opposed second end 54. The first end 52 of the fixation bracket 50 is coupled to the exterior side face 34 with the second end 54 projecting downward past the lower end face 32 of the body 30. The general shape of the fixation bracket 50 is contoured to align with an outer surface 56 (see FIGS. 1-2) of the tibia 16. A plurality of holes 58 are positioned along the length of the bracket 50 to enable the bracket 50 to be fixed to the body 30 of the prosthetic device 12 as well as to the tibia 16 itself. Any suitable fastener or adhesive can be used to couple the bracket 50 to the body 30 and tibia 16. However, in the present example, bone screws 60 are suitably used (see FIGS. 1-2).

Referring again to FIGS. 1-2, the prosthetic device 12 is configured to replace only a portion of the tibia 16. Thus, only a portion of the lateral condyle 24 is replaced by the prosthetic device 12 with the modular bearing plate 46 replacing a portion of the tibial plateau 28. Moreover, the body 30 is configured to extend only partially into the interior of the tibia 16. In this way, the prosthetic device addresses a number of the difficulties associated with traditional treatments for tibial plateau fractures involving fixation plates and screws to rebuild and/or repair the joint. In one aspect, the prosthetic device 12 is designed to replace the damaged portions of the bone rather than to repair it by creating immediate structural integrity and weight-bearing capabilities.

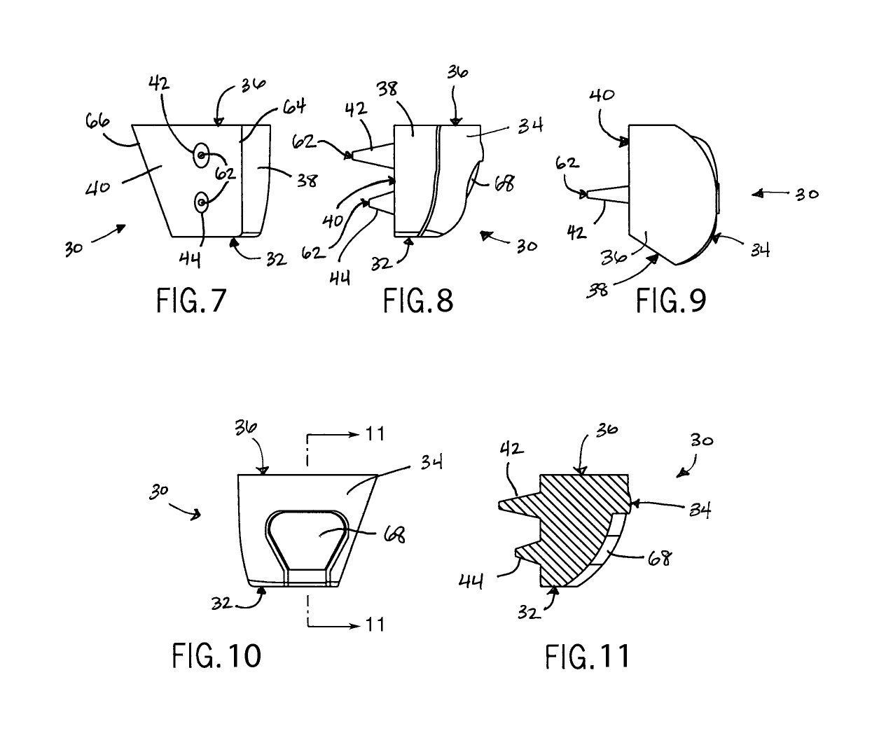

FIGS. 7-11 detail views of the body 30 of the prosthetic device 12. With reference to FIG. 7, a view of the interior faces 38 and 40 illustrates the conical projections 42, 44 projecting out of the plane of the page. A transverse cross-section of the conical projections 42, 44 has an oval or elliptical shape with a vertical dimension being greater than a horizontal dimension. In addition, it can be seen from FIGS. 7-9 that the ends 62 of the conical projections 42, 44 do not converge at point, but rather, each of the ends 62 defines a generally flat surface such that conical projections 42, 44 can be characterized as frustoconical.

FIGS. 8 and 9 also illustrate that the length of the first conical projection 42 in the axial direction is greater than the length of the second conical projection 44. In particular, FIG. 9 shows a plan view of the body 30 in which only the first conical projection 42 is visible due to the smaller size of the second conical projection 44. Thus, it will be appreciated that is not necessary for the conical projections 42, 44 to have the same dimensions. For example, the shape, size and angle of projection of an element (e.g., conical projections 42, 44) disposed on one of the interior faces 30, 40 can be tailored to the patient to be treated. Factors that can influence such elements can include the dimensions of the bone in which the prosthetic device 12 will be implanted, the characteristics of the fracture, the height, weight and/or age of the patient, and the material(s) used to fabricate the body 30.

FIGS. 7-11 further detail the dimensions of the faces of the body 30. For example, interior surface 40 can be seen to have a generally vertical edge 64 defining an intersection with interior surface 38 and an angular edge 66 such that the lower end face 32 at the base of the body 30 has a smaller horizontal cross-section than the upper end face 36 at the top of the body 30. With reference to FIGS. 10-11, the exterior side face 34 of the body 30 further includes a recess 68 shaped to receive the first end 52 of the fixation bracket 50. The recess 68 is formed in a central portion of the exterior side face 34 and extends downward with an opening in the lower end face 32. The design of the recess 68 enables the fixation bracket 50 to be seated such that an exterior surface 40 of the first end 52 is generally flush with the exterior side face 34. The fixation bracket 50 continues within the recess 68 out of the lower end face 32 of the body 30.

In one embodiment of a prosthetic device 12, the body 30 is at least partially composed of a porous material for promoting and/or supporting osteogenesis. The porous material can be disposed on the surfaces of the body 30 or make up an internal portion of the body 30. In one aspect, generally all of the body 30 is made up of the porous material. One example of a suitable porous material includes a porous metal structure with an average pore size of at least about 300 μm. The metal structure preferably has a high coefficient of friction (e.g., greater than about 0.9) and has interconnecting pores with at least about 70% porosity in order to promote bone in-growth (e.g., from the tibia into adjacent portions of the implanted body 30). In one aspect, the metal structure is designed to approximate cancellous or trabecular bone in terms of structure, function, and/or physiology. Preferably, the porous metal structure is also chemically stable and biologically inert. One non-limiting example material is a porous metallic material, also known as metal foam, which can be produced with interconnective porosity coupled with a regular pore shape and size. One such material that is currently commercially available for a variety of orthopedic implants involves the elemental metal tantalum fabricated with >80% interconnective porosity with use of a metal vapor deposition technique (Trabecular Metal from Zimmer, Warsaw, Ind., USA). This type of material is described in U.S. Pat. No. 5,282,861. While tantalum is one example porous metallic material, other non-limiting example porous metallic materials include titanium alloys, cobalt-chromium alloys, stainless steel alloys, tantalum alloys, and niobium alloys.

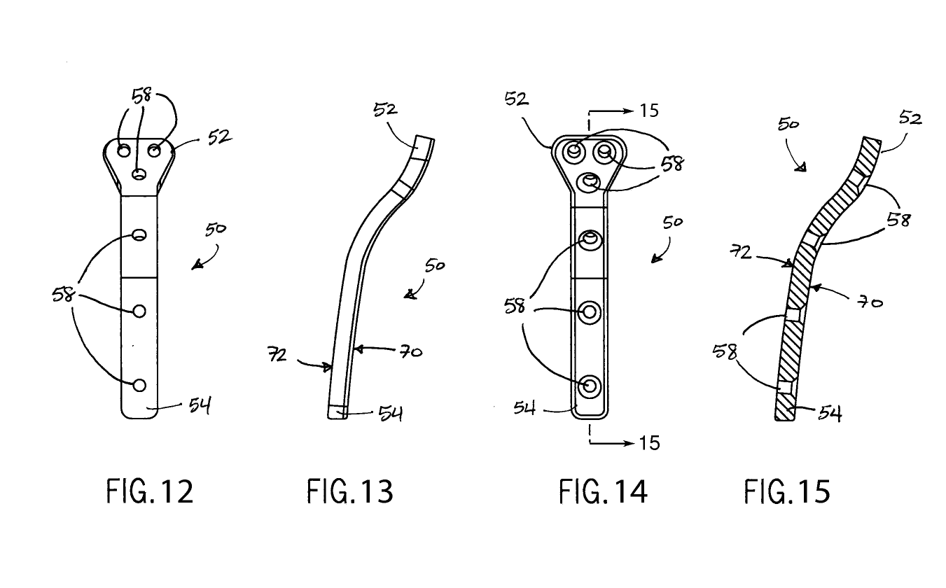

FIGS. 12-15 show schematic illustrations of the fixation bracket 50 in isolation. In the present example embodiment, the fixation bracket 50 has a solid, unitary construction selected from materials such as stainless steel, cobalt base alloys, bioceramics, titanium alloys, pure titanium, and polymers. A group of three holes 58 are positioned in the first end 52 and are spaced relative to one another such that the holes 58 lie at the points of a generally equilateral triangle. Three additional holes 58 are spaced along the length of the fixation bracket 50. From FIG. 15, it can be seen that four of the holes 58 are generally equally spaced apart along a central line extending between the first and second ends 52, 54 of the fixation bracket 50. In addition, each of the holes 58 passes between the exterior surface 70 and the interior surface 72 of the fixation bracket 50. The holes 58 define cylindrical passages with chamfered countersinks formed in the exterior surface 70 in order to accommodate the heads of screws 60.

The interior surface 72 of the fixation bracket 50 is configured to rest against an exterior surface of an anatomical part. In the present example, the interior surface 27 curves inwards from the first end 52 in order to rest against the outer surface 56 of the tibia 16. In one aspect, the shape, curvature, composition and length of the fixation bracket 50 can be modified to accommodate a specific patient or treatment. For example, in the case of larger or smaller bones, it can be preferable to increase or reduce the overall size of the bracket. Similarly, it can be preferable to include more or less than the six holes 58 shown in the Figures. In the case of a fixation bracket 50 with a reduced overall length, it can be desirable to either use fewer holes 58. It can also be preferable to omit the fixation bracket 50 entirely, for example, when the body 30 is fastened or adhered directly at the location of the implant.

A prosthetic device 12 according to the present invention can also include more than one fixation bracket 50. In one aspect, the body 30 can include recesses in addition to recess 68 in order to accommodate the use of an additional fixation bracket. Alternatively, the recess 68 can be extended upward to open to the upper end face 36. In this case, a central portion of a fixation bracket can be coupled to the body 30, such that a first end of the fixation bracket extends upwards past the upper end face 36 and a second end extends below a lower end face 32 of the body 30. In other embodiments, the body 30 does not include a recess 68 and the fixation bracket 50 is coupled directly to an outer surface of the body 30. In yet another aspect, the fixation bracket 50 and the body 30 can be fabricated as a single unit.

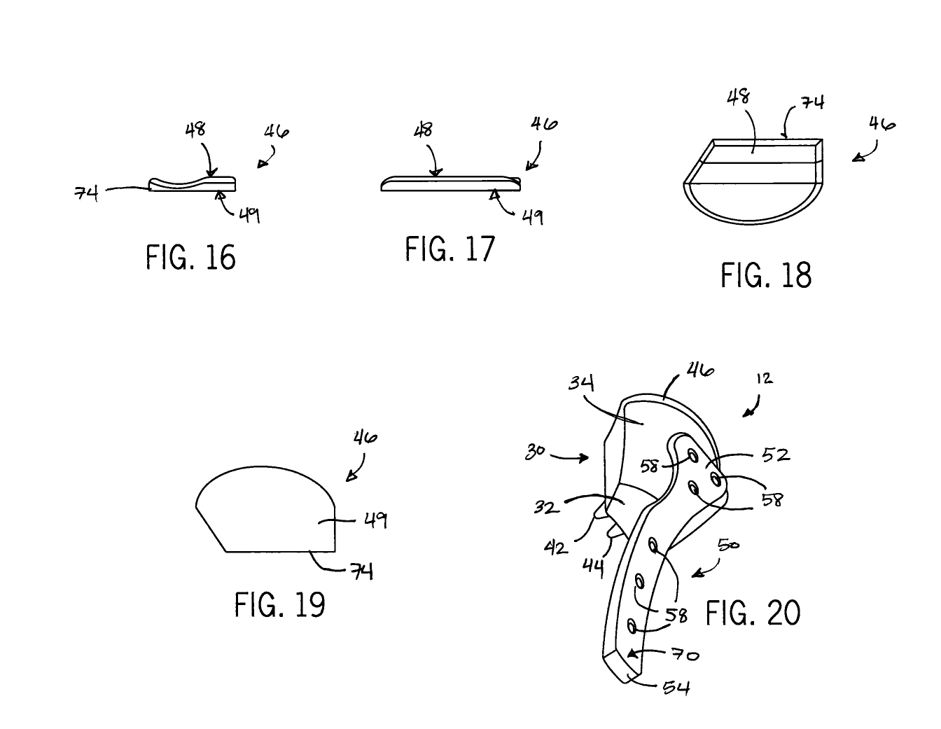

Turning now to FIGS. 16-19, aspects of the modular bearing plate 46 are schematically illustrated. The modular bearing plate 46 is generally shaped to correspond to and align with the outer perimeter of the upper end face 36 of the body 30. From FIG. 16, the articular surface 48 includes a beveled edge on the perimeter of the articular surface 48 and a slight depression that extends across the width of the modular bearing plate 46 in a direction parallel to an interior edge 74. The topography of the articular surface 48 is configured to function as an articulating surface for the lateral condyle 18 of the femur 14. Depending upon the location at which the prosthetic device is to be implanted, the topography of the articular surface 48 can be modified to maintain the efficacy of the modular bearing plate 46 as an articular surface. By contrast, the mounting surface 49 as shown in FIG. 19 is characterized by a generally flat surface with no bezel or recess. However, the mounting surface 49 can be modified to provide a suitable interface between the modular bearing plate 46 and the body 30. In one example, holes are provided in the modular bearing plate 46 and the body 30 to enable the use of screws to fasten the modular bearing plate 46 to the body 30.

The overall size and shape of the modular bearing plate 46 can also be modified to accommodate the nature of the procedure and the patient. For example, depending on the size and shape of the joint or the extent of the injury, the dimensions of the modular bearing plate 46 can be varied. In one aspect, the dimensions of the modular bearing plate 46 can be larger or smaller than the dimensions of the body 30. Thus, for example, the interior edge 74 can extend past the interior face 40 of the body 30 to provide a greater articular surface 48 to further augment the tibial plateau 28. In one embodiment, the modular bearing plate 46 is dimensioned to overlay generally the entire surface of the tibial plateau 28.

The modular bearing plate 46 can be fabricated from any suitable material. Preferably, the material used is selected from very hard alloys comprised of various combinations of aluminum, chromium, cobalt, iron, magnesium, molybdenum, nickel, and vanadium. More preferably, the material is selected from an alloy of cobalt and chromium. In one aspect, cobalt-chrome alloys facilitate smooth joint articulation and provide sufficient weight bearing capabilities. Polymeric materials such as ultra high molecular weight polyethylene may also be suitable for the modular bearing plate 46. While in one aspect, the modular bearing plate 46 is fabricated as a distinct component of the prosthetic device, in another aspect, the body 30 and modular bearing plate 46 can be fabricated as a single component.

In some embodiments of a prosthetic device 12 according to the present invention, the modular bearing plate 46 is removable from the body 30. The method of separating the modular bearing plate 46 from the body 30 can include removing one or more fastener used to couple the two components together, using a tool to pry the modular bearing plate 46 from the upper end face 36 of the body 30, cutting or sawing at the interface between the components, or any other suitable method. Preferably the modular bearing plate 46 can be removed from the body 30 after the prosthetic device 12 has been implanted in the patient for a period of time. In one aspect, where a patient develops arthritis or another condition requiring further treatment of the knee joint at a later date, a standard knee replacement can be implanted without removing the prosthetic device 12. Instead, the modular bearing plate 46 of the device 30 can be removed to enable implantation of a total knee replacement device.

A method of implanting a prosthetic device for partial replacement of an anatomical part can include preparing the patient for implantation of the prosthetic device, assembling portions of the prosthetic device prior to implantation, implanting and assembling portions of the device in the patient, and optionally, removing portions of the prosthetic device at a later date. In one example method, it can be preferable to resect at least a portion of the anatomical part in which the prosthetic device is to be implanted. In the case of a tibial plateau fracture, a portion of the tibia can be resected, such as a fractured portion of the lateral condyle. The prosthetic device and, more particularly, the body can then be implanted in the resected portion such that a lower end face of the body is supported on a cortical surface or other suitable load bearing surface of the anatomical part. An upper articular surface of a modular bearing plate supported on the body can be positioned to support articulation of an anatomical surface. The body can then be fixed to the anatomical part with the fixation bracket by fastening the bracket to an outer surface of the anatomical part with screws, for example.

It will be appreciated that embodiments of prosthetic devices according to the present disclosure are distinct from current prosthetic devices used in total joint replacement treatments. Total knee arthroplasties typically include the removal of both the distal end of the femur and the proximal end of the tibia. Both the cartilage and the anterior cruciate ligament are removed and the posterior cruciate ligament may also be removed. A device including femoral and tibial members is then implanted in the knee. By contrast the devices and methods of the present invention can be used to replace only a portion of the joint thereby leaving a substantial majority of the bones, cartilage and ligaments intact. In the example embodiment shown in the Figures, the prosthetic device 12 is used to replace only a portion of the lateral condyle 24 of the tibia 16. Alternatively, in the case where a fracture has occurred in the medial condyle 26, an embodiment of a prosthetic device could be used to replace only the damaged portion of the medial condyle 26. It will be further appreciated that embodiments of the present disclosure can be used to treat fractures in other anatomical parts including, for example the femoral portion of the knee joint.

The present invention has been described in terms of one or more preferred embodiments, and it should be appreciated that many equivalents, alternatives, variations, and modifications, aside from those expressly stated, are possible and within the scope of the invention. Moreover, while present inventive concepts have been described with reference to particular embodiments, those of ordinary skill in the art will appreciate that various substitutions and/or other alterations may be made to the embodiments without departing from the spirit of present inventive concepts. Accordingly, the foregoing description is meant to be exemplary, and does not limit the scope of present inventive concepts.

In addition, a number of examples have been described herein. Nevertheless, it should be understood that various modifications may be made. For example, suitable results may be achieved if the described techniques are performed in a different order and/or if components in the described system, architecture or device are combined in a different manner and/or replaced or supplemented by other components or their equivalents. Accordingly, other implementations are within the scope of the present inventive concepts. In particular, the Figures illustrate one example embodiment of a prosthetic device for partial replacement of a knee joint. However, embodiments of prosthetic devices can be used for the treatment of other joints or anatomical parts such as wrist joints, hip joints, ankle joints and the like. Note also that each reference identified in the present application is herein incorporated by reference in its entirety.

What is claimed is:

1. A prosthetic implant for partial replacement of an anatomical part, the implant comprising:
a unitary body including a lower end face for supporting the body on a cortical surface, a generally curved exterior side face and an upper end face;
a modular bearing plate disposed on the upper end face, the bearing plate having an upper articular surface for supporting articulation of an anatomical surface and an opposed mounting surface in contact with the upper end face; and
a fixation bracket with a first end and an opposed second end, the first end of the fixation bracket coupled to the exterior side face and the second end projecting downward past the lower end face of the body, the fixation bracket having a contour generally corresponding to an outer surface of an anatomical part,
wherein the prosthetic implant is configured to replace a portion of the outer surface of the anatomical part and extend partially into the anatomical part.

2. The prosthetic implant of claim 1, wherein the body further includes at least one projection extending laterally from an interior side face of the body.

3. The prosthetic implant of claim 2, wherein the at least one projection comprises a first projection and a second projection, and wherein the first and second projections have different lengths.

4. The prosthetic implant of claim 3, wherein the first and second projections have a generally conical shape that tapers away from the interior side face of the body.

5. The prosthetic implant of claim 1, wherein the exterior side face of the body further includes a recess shaped to receive a first end of the fixation bracket.

6. The prosthetic implant of claim 1, wherein the body comprises a porous material capable of supporting osteogenesis.

7. The prosthetic implant of claim 6, wherein the porous material comprises a metal with an average pore size of at least about 300 μm and a porosity of at least about 70%.

8. The prosthetic implant of claim 1, wherein the modular bearing plate comprises an alloy of cobalt and chromium.

9. The prosthetic implant of claim 1, wherein the fixation bracket further comprises at least one hole formed in the bracket.

10. The prosthetic implant of claim 1, wherein the modular bearing plate is removable from the body.

11. A knee joint prosthetic implant for partial replacement of a proximal portion of the tibia, the implant comprising:
a unitary body including a lower end face for supporting the body on a cortical surface of the tibia, a generally curved exterior side face and an upper end face;
a modular bearing plate disposed on the upper end face, the bearing plate having an upper articular surface for supporting articulation of a condyle surface of a femur and an opposed mounting surface in contact with the upper end face; and
a fixation bracket with a first end and an opposed second end, the first end of the fixation bracket coupled to the exterior side face and the second end projecting downward past the lower end face of the body, the fixation bracket having a contour generally corresponding to an outer surface of the tibia,
wherein the prosthetic implant is configured to replace a portion of the proximal end of the tibia including at least a portion of the tibial plateau, and extend partially into the tibia.

12. The prosthetic implant of claim 1, wherein the body further includes at least one projection extending laterally from an interior side face of the body.

13. The prosthetic implant of claim 11, wherein the exterior side face of the body further includes a recess shaped to receive a first end of the fixation bracket.

14. The prosthetic implant of claim 11, wherein the body comprises a porous material capable of supporting osteogenesis.

15. The prosthetic implant of claim 11, wherein the modular bearing plate comprises an alloy of cobalt and chromium.

16. The prosthetic implant of claim 11, wherein the modular bearing plate is removable from the body.

17. A method of implanting a prosthetic implant for partial replacement of an anatomical part, the method comprising:
resecting a portion of the anatomical part;
implanting a unitary body in the resected portion, the body including a lower end face for supporting the body on a cortical surface of the anatomical part, a generally curved exterior side face and an upper end face;
supporting a modular bearing plate on the body, the modular bearing plate disposed on the upper end face, the bearing plate having an upper articular surface for supporting articulation of an anatomical surface and an opposed mounting surface in contact with the upper end face; and
fixing the body to the anatomical part with a fixation bracket, the fixation bracket comprising a first end and an opposed second end, the first end of the fixation bracket coupled to the exterior side face and the second end projecting downward past the lower end face of the body, the fixation bracket having a contour generally corresponding to an outer surface of the anatomical part,
wherein the prosthetic implant is configured to replace a portion of the outer surface of the anatomical part and extend partially into the anatomical part.

18. The method of claim 17, wherein the prosthetic implant of claim 1, wherein the body further includes at least one projection extending laterally from an interior side face of the body.

19. The method of claim 17, wherein the body comprises a porous material capable of supporting osteogenesis.

20. The method of claim 17, further comprising one of removing and replacing the modular bearing plate.
